(19) United States
(12) Patent Application Publication
(10) Pub. No.: US 2005/0008677 A1
(43) Pub. Date: Jan. 13, 2005

(54) DELIVERY SYSTEM OF HOMOGENEOUS, THERMOREVERSIBLE GEL FILM CONTAINING KAPPA-2 CARRAGEENAN

(75) Inventors: James Modliszewski, Brick, NJ (US); Arthur Ballard, Westport, ME (US); Christopher Sewall, Hope, ME (US); William Blakemore, Topsham, ME (US); Peter Riley, Yardley, PA (US)

Correspondence Address: US

(73) Assignee: FMC Corporation, Philadelphia, PA (US)

(21) Appl. No.: 10/824,793

(22) Filed: Apr. 14, 2004

Related U.S. Application Data
(60) Provisional application No. 60/462,794, filed on Apr. 14, 2003.
(60) Provisional application No. 60/462,758, filed on Apr. 14, 2003.
(60) Provisional application No. 60/462,721, filed on Apr. 14, 2003.
(60) Provisional application No. 60/462,617, filed on Apr. 14, 2003.
(60) Provisional application No. 60/462,785, filed on Apr. 14, 2003.
(60) Provisional application No. 60/462,783, filed on Apr. 14, 2003.
(60) Provisional application No. 60/462,792, filed on Apr. 14, 2003.
(60) Provisional application No. 60/462,793, filed on Apr. 14, 2003.

(51) Int. Cl. A61K 47/00

(52) U.S. Cl. 424/439

(57) ABSTRACT

Abstract of the Disclosure

The present invention is directed to a delivery system comprising a homogenous, thermoreversible gel film, wherein the gel film comprises: (i) a film forming amount of kappa-2 carrageenan and optionally at least one of a plasticizer, a second film former, a bulking agent, and a pH controlling agent; and (ii) an active substance. The present invention is also directed to a process for the manufacture thereof.

### Detailed Description of the Invention

### RELATED APPLICATIONS

This application claims the benefit of U.S. Provisional Application No. 60/462,794, filed on April 14, 2003.

### FIELD OF THE INVENTION

The present invention is directed to a delivery system comprising a homogenous, thermoreversible gel film, wherein the gel film comprises: (i) a film forming amount of kappa-2 carrageenan and optionally at least one of a plasticizer, a second film former, a bulking agent, and a pH controlling agent; and (ii) an active substance. The present invention is also directed to a process for the manufacture thereof.

### BACKGROUND OF THE INVENTION

Use of delivery systems, for example, for oral care actives in breath and cleansing strips, has increased over recent years. The present invention generally relates to delivery systems comprising a gel film that can be used to contain and deliver a wide variety of active substances.

For example, WO 02/43657 discloses the use of pullulan free edible film compositions containing at least one film forming agent, at least one bulk filler agent, at least one plasticizer. These films can contain a number of materials such as medicaments and can be used for a number of applications in oral care; e.g., breath freshening strips. The film forming agent is widely described as being any of cellulose ethers (stated to include: methylcellulose, ethylcellulose, hydroxymethylcellulose, hydroxyethylcellulose, hydroxypropylmethylcellulose; carboxymethylcellulose and derivatives thereof); modified starches (stated to include acid and enzyme hydrolyzed corn and potato starches); natural gums (stated to include gum Arabic, guar gum, locust bean gum, carrageenan gum, acacia, karaya, ghatti, tragacanth agar, tamrind gum, xanthan gum and derivatives thereof); edible polymers (stated to include microcrystalline cellulose, cellulose ethers, xanthan and derivatives thereof); hydrocolloid flours (stated to include guar gum, locust bean, microcrystalline cellulose, tara and derivatives thereof); seaweed extracts (stated to include alginate, carrageenan and derivatives thereof); and land plant extract (stated to include konjac, pectin, arabinoglactan and derivatives thereof). There is no mention of any problems associated with making carrageenan based gel films, how to overcome such problems, that carrageenans vary in type, compositional make-up and properties, or the use, particularly, of kappa-2 carrageenan.

WO 00/18365 discloses fast dissolving orally consumable films used to deliver breath deodorizing agents, antimicrobial agents and salivary stimulants to the oral cavity. The film forming agent to be used in making the film of the invention is said to be selected from pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, Arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, casein and mixtures thereof. These films are said to overcome the challenge of maintaining the essential oil interaction and relatively high oil content in certain films sold for oral care purposes. There is no mention of carrageenan or, particularly, the use of kappa-2 carrageenan.

### SUMMARY OF THE INVENTION

As a first embodiment, the present invention is directed to a delivery system comprising a homogenous, thermoreversible gel film, wherein the gel film comprises: (i) a film forming amount of kappa-2 carrageenan and optionally at least one of a plasticizer, a second film former, a bulking agent, and a pH controlling agent; and (ii) an active substance.

As a second embodiment, the present invention is directed to a process for preparing the homogeneous gel film delivery system described immediately above comprising the steps of: (i) heating, hydrating, mixing, solubilizing and, optionally, de-aerating the kappa-2 carrageenan and optionally at least one of the plasticizer, the second film former, the bulking agent, and the pH controlling agent in an apparatus providing sufficient shear, temperature and residence time to form a homogeneous molten composition, wherein the temperature is at or above the solubilizing temperature of the composition; (ii) adding an effective amount of an active substance either prior to or after formation of the molten composition; and (iii) cooling the molten composition containing the active substance at or below its gelling temperature to form the gel films containing the active substance.

### BRIEF DESCRIPTION OF THE DRAWINGS

Figure 1 is a partially broken away, side elevational view of the fluid mixing apparatus for mixing first and second fluids with steam that can be used in the process of the present invention.

### DETAILED DESCRIPTION OF THE INVENTION

Carrageenan is a commercially significant galactan polysaccharide found in red seaweed. All carrageenans contain repeating galactose units joined by alternating α1→3 and β1→4 glycosidic linkages and are sulfated to widely varying degrees. The types of carrageenan may be distinguished, in part, by their degree and position of sulfation, as well as the seaweed from which they are obtained. For example, iota carrageenan has a repeating unit of D-galactose-4-sulfate-3,6-anhydro-D-galactose-2-sulfate providing a sulfate ester content of about 25 to 34%. Iota carrageenan can be obtained, for example, from Eucheuma denticulatum (also known as "Spinosum") . Kappa carrageenan has a repeating unit of D-galactose-4-sulfate-3,6-anhydro-D-galactose and is obtained, for example, from Kappaphycus alvarezii (also known as " Eucheuma cottonii") . In contrast, kappa-2 carrageenan is reported by R. Falshaw, H.J. Bixler and K. Johndro, Structure and Performance of Commercial Kappa-2 Carrageenan Extracts , Food Hydrocolloids 15 (2001) 441-452, and by H. Bixler, K Johndro and R Falshaw, Kappa-2 carrageenan: structure and performance of commercial extracts II , Food Hydrocolloids 15 (2001) 619-630 to be copolymers containing a certain amount of kappa repeating units (3:6-anydroglactose (3:6-AG)) and iota repeating units (3:6-anhydroglactose-2-sulfate (3:6-AG-2-S)) covalently bound in the copolymer backbone and obtained from certain Gigartinaceae algae. The foregoing references state that such kappa-2 carrageenans have distinctly different properties as compared to simple mixtures of kappa and iota carrageenans. Other references discussing kappa-2 carrageenan are discussed in these publications. Kappa-2 carrageenan extracted from Gigartina atropurpurea is reported by R. Falshaw, H Bixler and K Johndro, Structure and Performance of Commercial Kappa-2 Carrageenan extracts III , Food Hydrocolloids 17 (2003) 129-139. While there has been considerable confusion historically about the physical nature of kappa-2 carrageenans, recent studies, such as those mentioned immediately above, have confirmed that kappa-2 carrageenans are copolymers containing kappa and iota repeating units covalently bound (in certain ratios of kappa to iota moieties) in the copolymer backbone in clear distinction to physical mixtures of kappa and iota polymers.

As used herein, kappa-2 carrageenan has a molar ratio of 3:6AG-2S to 3:6AG content of 25 to 50%, iota carrageenan has a molar ratio of 3:6AG-2S to 3:6AG content of 80 to 100% and kappa carrageenan has a molar ratio of 3:6AG-2S to 3:6AG content less than that for kappa-2 carrageenan. For example, kappa carrageenan from Eucheuma cottonii , a commonly known and used seaweed source for kappa carrageenan, has a molar ratio of 3:6AG2S to 3:6AG content of less than about 10%; and iota carrageenan from Spinosum , a commonly known and used seaweed source for iota carrageenan, has a molar ratio of 3:6AG2S to 3:6AG content greater than about 85%. This means that kappa-2 carrageenan comprises a ratio of kappa (3:6-AG) to iota (3:6-AG-2-S) repeating units between 1.0 to 3.0:1, more particularly, 1.5 to 3.0:1 (more particularly depending on the desired application). The molar ratio of 3:6AG-2S to 3:6AG content of 25 to 50% holds in kappa-2 carrageenans regardless of its degree of modification and precursor content (e.g., mu and nu repeating units). Thus, any kappa-2 carrageenan meeting the molar ratio of 3:6AG-2S to 3:6AG content of 25 to 50%, regardless of its degree of modification, is within the scope of this invention.

The kappa-2 carrageenan to be used in the present invention may be contained within or purified or separated from a number of seaweed species within the class of, for example, Gigartinaceae algae such as Gigartina radula, Gigartina corymbifera, Gigartina skottsbergii, Iridaea cordata, Sarcothalia crispata, and Mazzaella laminarioides. The seaweed source of the kappa-2 carrageenan to be used in this invention is any that produces kappa-2 carrageenan having the molar content of 3:6AG-2S to 3:6AG described herein. The kappa-2 carrageenan that can be used in the present invention may occur naturally in the seaweeds above or may be modified from the above seaweeds to increase the amount of 3:6AG-2S and 3:6AG moieties in the kappa-2 carrageenan from their precursors (e.g., 3:6AG-2S moiety within the kappa-2 carrageenan modified from its precursor nu upon alkali treatment, and 3:6AG moiety within the kappa-2 carrageenan modified from its precursor mu upon alkali treatment). The recovery and modification techniques are well known in the art including the cited publications by Falshaw, Bixler and Johndro. For example, modification of the kappa-2 carrageenan can occur during its recovery from certain Gigartinacean algae as a result of alkali treatment at elevated temperatures. Recovery methods include the optional full or partial filtration of insolubles from the starting material or the use of unfiltered material. When the nu and mu precursors in the kappa-2 carrageenan are modified to 3:6AG-2S and 3:6AG, respectively, such modification may be complete (i.e., 100% of the nu and mu precursors in the kappa-2 carrageenan are modified to 3:6AG-2S and 3:6AG moieties, respectively) or less than fully complete (i.e., less than 100% of the nu and mu precursors in the kappa-2 carrageenan are modified to 3:6AG-2S and 3:6AG moieties, respectively). It is understood that during the recovery process of the kappa-2 carrageenan from the above seaweeds small or trace amounts of other carrageenans (e.g., lambda carrageenan) may be present and such can be used with the kappa-2 carrageenans in the present invention.

One of the surprising aspects of the present invention is the functionality of the kappa-2 carrageenan as compared to kappa carrageenans, iota carrageenans and simple dry mixtures of kappa and iota carrageenans that contain the identical level of 3:6-AG-2-S. That is, iota and kappa carrageenans are gelling carrageenans. In distinction, kappa-2 carrageenans are known to be weakly gelling. As a result, it would have been expected that such weakly gelling carrageenans would form weak gel films. However, to the surprise of the Applicants, kappa-2 carrageenans have been found to form surprisingly strong films.

Without being bound, it is generally recognized that the water gel strength of kappa carrageenan decreases significantly as 3:6-AG-2-S content increases (e.g. 1,500g to 300g at 1% in water), this being due to the structural interference of these additional ester sulphates with helical aggregation and hydrogen bonding between such aggregated helices. This trend continues for kappa-2 (25-50%, more particularly, 25-40% 3:6-AG-2-S for particular applications), with water gels as low as 150g and is likely due to its structural variability. Iota carrageenan (e.g., 80 to 100% 3:6-AG-2-S), however, is more ordered structurally, thereby contributing a more uniform three dimensional structure to this water gel, providing a stronger water gel, as indicated by its rupture strength of over 300g. While not bound by any theory, it is thought that simple physical mixtures of kappa and iota carrageenans are somewhat antagonistic with respect to gel strength, most likely due to mutual interference of their ideal gel structures developed at separate temperatures upon cooling. Resultant gel strength values for the dry blended, physical mixtures of kappa and iota carrageenan based water gels are still much higher in water gel strength than kappa-2 carrageenan. One can also achieve this antagonistic effect by separately hydrating & solubilizing the kappa and iota carrageenans, and, while maintaining their solutions above their gelling temperatures, uniformly combine solutions and cast or allow the blend to cool to initiate gelation. This gel strength drop (structural weakening) is further aggravated by extract viscosity reduction (shorter molecules) and divalency addition. Thus, based on traditional gel strength and textural measurement, kappa-2 carrageenan would not be expected to be appropriate for gel film applications. However, as the inventors here found, when kappa-2 is applied to making gel films, it demonstrates surprising film strength and mechanical integrity, well beyond expectations based on traditional (prior art) molecular structuring with respect to water gels. It also demonstrates full compatibility with traditional film ingredients, such as starch, humectant, etc. It is thought that the random copolymeric gel structure of kappa-2 in such gel films and film compositions is ideal contributing complete structural stability from the onset of gelation, with no need or tendency to change over time or during film drying process. The structure stays as it is gelled, unlike kappa carrageenan structure that continues to harden, iota carrageenan that is too elastic and won't tighten, and kappa/iota physical blends (as opposed to kappa-2 copolymers) that exhibit structural interferences. This surprising film strength of kappa-2 carrageenan also allows carrageenan molecular weight control in order to better balance process viscosity and required film strength for mechanical processing, such direction resulting in the capability to operate at lower moisture levels in the cast films while maintaining other essential film properties.

The kappa-2 carrageenan is used in the present invention in a film forming amount (e.g., an amount that adds film strength to the gel film) which is distinguished from trace amounts of kappa-2 carrageenan that do not add film properties to the film. Thus, for example, in a gel film of the present invention containing the second film formers discussed below, a film forming amount of kappa-2 carrageenan is an amount that adds film strength to the overall film. Such film forming amounts are generally at least 0.5% by weight of the dry gel film, particularly, 0.5% to 90%, more particularly, 0.5% to 50%, more particularly, 0.5% to 25%, more particularly, 1.5% to 25% by weight of the dry gel film depending on the desired characteristics.

As used herein, "homogeneous film" defines films that, to the naked eye, are visually uniform and free of defects such as lumps, cracks, particles that are undissolved that should be dissolved, non-uniform distribution of insoluble particles, etc. "Fish eyes" (mixed liquid and solid states) or "gel balls" (non-uniform gel structure) would not meet the definition of "homogeneous" as used herein.

The gel films of the present invention are homogeneous, thermoreversible gel films. They can be cast and used in a wide variety of applications as cast films.

As used herein, "thermoreversible film" defines a film that has a melting temperature. As used herein, the melting temperature is the temperature or temperature range over which the gel film softens or flows.

As used herein, the phrase "gel films" refer to a thin membrane or three dimensional network, formed from structured kappa-2 carrageenan. The gel-forming composition is characterized by a gel temperature, the temperature below which the molten mass of the gel composition must be cooled to form a self-supporting structure. Optionally, a molten mass can be cast hot and allowed to cool, as well as dry to further concentrate the solids (controlled moisture removal) until a gel film is formed by the gel composition. The melt temperature of a thermoreversible gel film is higher than its gel temperature.

The gel film of the present invention includes an active substance. Examples of active substances that may be contained within the gel film is at least one of an oral care agent, a breath freshening agent, an antimicrobial agent, a cooling agent, a pharmaceutical agent, a nutraceutical agent, a salivary stimulant agent, a vitamin, a mineral, a coloring agent, cosmetic ingredient, agricultural active, a sweetener, a flavorant, a fragrance or a food. Examples of a flavorant include sugar, corn syrup, fructose, sucrose, aspartame, sucrolose, sorbitol, mannitol, maltitol, etc, whether or not other components, such as plasticizers, bulking agents, second film formers, etc. are present. One embodiment of a delivery system of the invention comprises kappa-2 carrageenan, flavorant and water in a high solids system; e.g., greater than 50%, 60%, 65%, 75%, 80%, 85%, 90% solids.

The gel film of the present invention desirably contains soluble gelling cations that promote carrageenan structure formation; i.e., gel formation. Such beneficial cations include potassium, sodium and ammonium. These cations can be present within the kappa-2 carrageenan or added to it from other organic or inorganic sources, at various points in the process, while maintaining the molten mass above its gelation temperature. These beneficial cations can be present in an amount of less than 20% by dry weight of the kappa-2 carrageenan in the gel film (including water). This amount can be varied depending on the components in the system, desired melt and sealing temperatures, and processing conditions and equipment choices.

Other soluble cations, such as calcium, magnesium, aluminum and chromium can adversely impact stability and should be kept to a minimum, such as less than 10%, less than 5%, less than 1% by dry weight of the kappa-2 carrageenan in the gel film (including water). Sequestering or chelating agents could be added in sufficient amounts to minimize the above cation solubility (and participating activity), providing the gel system is not adversely affected by the sequestering agent nor the resulting compound.

The molecular weight of the kappa-2 carrageenan is generally above 100,000 Daltons, preferably, 100,000 to 1,000,000 Daltons, more preferably, 100,000 to 450,000, more preferably, 100,000 to 350,000 depending on the application.

In some film applications of the present invention, reducing the gelling temperature of the kappa-2 carrageenan is desirable. A gelled system of kappa-2 carrageenan having an average to high molecular weight has a gelling temperature of at least 59°C and 35°C in the potassium/calcium and sodium forms, respectively. Thus, replacing the potassium cation with sodium cation is one way to reduce the gelling temperature of kappa-2 carrageenans. It has generally been considered that the gelling temperature is independent of the molecular weight of the kappa-2 carrageenans. However, surprisingly, Applicants have further discovered that in high solids systems having at least 50% solids, using a kappa-2 carrageenan having a reduced molecular weight (e.g., having a viscosity of 19 cps or less, more particularly less than 10 cps, at 75 °C when measured in a 0.10 molar sodium chloride solution containing 1.5% of the reduced molecular weight carrageenan by weight based on the total weight of the solution; this viscosity test can be performed using a Brookfield LVF (Brookfield Engineering Laboratories, Inc.) viscometer using Spindle #1 at 60 r.p.m. and determining the viscosity after six revolutions) can further reduce the gelling temperature of kappa-2 carrageenan, for example, from 35°C to 25°C in the sodium form and from 59°C to 57°C in the potassium/calcium form. Reducing the gelling temperature of the kappa-2 carrageenan generated structure can have beneficial effects in the processing of the gel films of the present invention, for example, by lowering the amount of heat used in the manufacturing process and minimizing residual stress on the dried film.

The homogeneous, thermoreversible gel film of the present invention can optionally contain at least one of a plasticizer, a second film former, a bulking agent and a pH controlling agent. The components to be added to the gel film and their amounts can vary depending on the desired use of the kappa-2 gel film delivery system.

Examples of such a plasticizer include polyols such as glycerin, sorbitol, maltitol, lactitol, corn starch, polydextrose, fructose, solubilized oils, and polyalkylene glycols such as propylene glycol and polyethylene glycol. The amount of the plasticizer can vary depending on the desired use and elasticity of the gel film delivery system. For example, such plasticizers can generally be used in an amount of at least 10%, preferably, at least 20%, more preferably, at least 30% by weight of all the components including water in the dry film depending on the desired elasticity of the delivery system. It is possible that the delivery system contains no plasticizer at all.

Examples of the second film former that can be used in the present invention include at least one of a hydrocolloid, an alkylcellulose ether or a modified alkyl cellulose ether. Examples of the hydrocolloid include at least one of kappa carrageenan; iota carrageenan; kappa and iota carrageenans having a reduced molecular weight (e.g., having a viscosity of 19 cps or less, more particularly less than 10 cps, at 75 °C in a 0.10 molar sodium chloride solution containing 1.5% of the reduced molecular weight carrageenan by weight of the total weight of the solution; this viscosity test can be performed using a Brookfield LVF (Brookfield Engineering Laboratories, Inc.) viscometer using Spindle #1 at 60 r.p.m. and determining the viscosity after six revolutions) and less than fully modified versions thereof; alginates including potassium alginate, sodium alginate, ammonium alginate and propylene glycol alginate; low viscosity polymannan gums (e.g., generally less than about 1000 mPs viscosity as measured at 1 wt % in water at 25 ^{o} C) such as low viscosity guar gum; pullulan, gellan (including high and low-acyl gellan); dextran, pectin and combinations thereof. An example of an alkylcellulose ether that can be used in the present invention is hydroxyethyl cellulose. Examples of modified alkyl cellulose ethers that can be used in the present invention include hydroxypropyl cellulose and hydroxypropylmethyl cellulose. The kappa-2 carrageenan can be the only film former in the gel film. When the gel films of the present invention contain second film formers, the kappa-2 carrageenan can be present in an amount of at least 10%, at least 40%, at least 60% or at least 80% by weight of the total amount of film formers in the dry gel film.

A dried film is the residual form of a cast film after controlled water removal. Combinations of ingredients, such as: kappa-2 carrageenan, and, optionally, a starch, a polyol and water for processing, are dispersed, hydrated, solubilized and, optionally, de-aerated within the process options described within. The resulting homogeneous mass is cast or formed at the desired solids level (necessary to achieve the intended end-product). The cast system is formed, via gravitational or controlled forces, and subsequently either immediately further processed or the cast mass is additionally processed by utilizing various methods for uniform and controlled water removal until the desired moisture level is reached. Controlled water removal from the cast system allows a further strengthening/alignment of the homogeneous film ingredients into a denser structure, which can further strengthen film characteristics. Moisture removal is limited to that moisture not bound to the molecular surface of the various hydrocolloid and carbohydrate components. The dried film is achieved when the originally cast film does not lose additional weight while subject to the various drying methods employed in the dewatering/dehydration process. A reduction in moisture content to constant levels also imparts stability to the film and, optionally, its contents (if embedded or enrobed or entrapped, etc.) as water activity is also reduced by the process.

Examples of the bulking agent include non-colloidal (vegetal sourced) cellulose, microcrystalline (vegetal sourced) cellulose, microcrystalline starch, modified and unmodified starch, starch derivatives and fractions, inulin, starch hydrozylates, sugar, corn syrup and polydextrose. As used herein and in the claims, the term "modified starch" includes such starches as hydroxypropylated starches, acid-thinned starches, and the like. Examples of modified starches that can be used in the present invention include Pure Cote™ B760, B790, B793, B795, M250 and M180, Pure-Dent™ B890 and Pure-Set™ B965, all available from Grain Processing Corporation of Muscatine, Iowa, and C AraTex™ 75701, available from Cerestar, Inc. Examples of starch hydrozylates include maltodextrin, also known as dextrin. Unmodified starches such as potato starch can also contribute to the film strength when combined with the hydrocolloids within the scope of the invention. In general, modified starches are products prepared by the chemical treatment of starches, for example, acid treatment starches, enzyme treatment starches, oxidized starches, cross-bonding starches, and other starch derivatives. It is preferred that the modified starches be derivatized wherein side chains are modified with hydrophilic or hydrophobic groups to thereby form a more complicated structure with a strong interaction between side chains.

The amount of the bulking agent to be used in the present invention is generally 0 to 20% by weight of the dry film, but more can be used, if desired, for example, at least 20%, more preferably, at least 30% by weight of the dry film depending on the use of the delivery system.

Note that starch, starch derivatives and starch hydrozylates can be multifunctional. That is, in addition to being used as bulking agents, they can be used as second film formers. When such are used as bulking agents and second film formers, they are generally used in an amount of at least 10%, preferably, at least 20%, by weight of _the dry gel film depending on the application.

Examples of the pH controlling agent that can optionally be used in the present invention include bases such as hydroxides, carbonates, citrates and phosphates, mixtures thereof and their salts (e.g., sodium citrate). The pH controlling agent can be chosen as the source of added beneficial cations such as potassium or sodium. For some compositions, the pH controlling agent can be used to improve the stability of the gel film. The amount of the pH controlling agent is generally in the amount of 0 to 4%, preferably, 0 to 2%.

The dry gel films (e.g., 80% solids or higher) of the present invention have been found to have, for example, a break force of at least 250 grams, at least 1,000 grams, at least 2,500 grams, at least 4,000 grams, at least 5,000 grams and at least 6,000 grams, as determined using a Texture Analyzer TA-108S Mini Film Test Rig. At lower solids, the gel films have been found to have a break force of at least 50 grams, at least 100 grams, at least 200 grams, as determined in a similar manner.

The films of the present invention have been found to have a solids content of at least 50%, at least 60%, at least 70%, at least 80% and at least 90% of all components in the gel film. It is understood that up to 15%, 10%, 5% water may remain strongly associated with the solids in the dry gel film.

It is possible that the films of the present invention can contain nonthermoreversible gums. However, so as not to adversely impact the homogeneous and thermoreversible nature of the gel films of the present invention, such nonthermoreversible gums should be present in an amount of less than 50% by weight of the kappa-2 carrageenan, preferably, less than 40% more preferably, less than 30%. Examples of such nonthermoreversible gums include crosslinked gums such as calcium set (e.g., crosslinked) pectins and/or alginates. Calcium reactive alginates and pectins, as well as their less refined forms, are considered as thermoreversible gums in the absence of divalent cations. Other non-thermoreversible gums such as tragacanth gum contribute to the thermoreversability of the kappa-2 carrageenan by absorption of water within its structure thereby causing the kappa-2 carrageenan to form a denser, three-dimensional structure, as it is solubilized in less water, providing the same effect as increasing the kappa-2 carrageenan amount without the secondary film formers. Additional film formers, such as polymannans can form continuous networks, either by themselves or synergistically with other components during the activation and casting process.

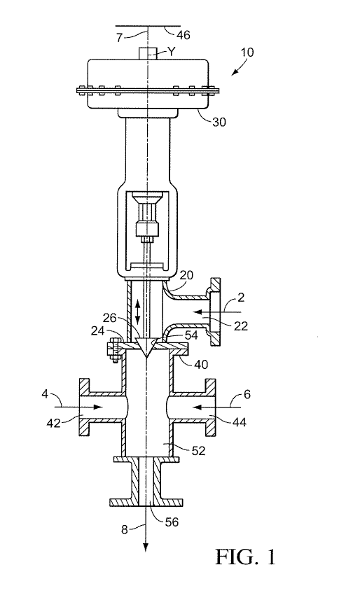

The kappa-2 carrageenan gel films of the present invention are generally made from a process utilizing an apparatus that enables sufficiently high shear, temperature (above the gelling temperature) and residence time so as to provide a homogeneous molten mass of the composition and formation of the gel upon cooling. Such apparatus include but are not limited to Ross mixers, Stephan processors, conventional jet cookers, extruders and the fluid mixing apparatus as set forth in Figure 1. Ross mixers, Stephan processors, extruders and conventional jet cookers are readily available commercially. Prior to cooling, the molten mass can be fed to at least one of a pump, mixer or devolatilizer. An example of a device that performs any one of such functions is an extruder. An extruded molten mass can also be directed to a film forming or shaping device (e.g. spreader box) that aids in the uniform casting of a continuous film, or, through a die that allows a direct formation of a film or shaped extrudate from the molten mass delivery equipment. Care must be taken to maintain the molten mass above the initiation of restricted flow/gel structure formation. Insulated and pre-heated (to maintain proper temperatures) transfer hoses may be used to insure molten mass flow until desired gel film formation is initiated. Additional processing methods (such as pre-heating the discharge/plunger-like head as seen in a Ross process system) can force (by pressure) the molten mass through the transfer hoses mentioned above. Additional insulation can help maintain molten mass temperatures through the use of a Teflon disk initially placed upon the molten mass surface immediately after removing the mixing device. Note, as an additional aspect of the invention, the molten mass need not necessarily reach full homogeneity in step (i). That is, when an additional apparatus such as a mixer, pump and/or devolatilizer is used, homogeneity of the molten mass can be obtained prior to or after feeding the molten composition into at least one of the mixer, pump or devolatilizer provided the molten mass reaches homogeneity prior to gelling.

Examples of the apparatus in step (i) include but are not limited to Ross mixers, Stephan processors, conventional jet cookers, extruders and the fluid mixing apparatus as set forth in Figure 1. Stephan processors, Ross mixers, extruders and conventional jet cookers are readily available commercially.

As used herein, the "fluid mixing apparatus" refers to FIG. 1. FIG. 1 illustrates a fluid mixing apparatus 10. The fluid mixing apparatus 10 is arranged to mix steam 2 with a first fluid or slurry 4 and a second fluid or slurry 6 to produce a molten mass or slurry mixture 8.

The fluid mixing apparatus 10 comprises a first housing 20 having a first inlet 22 through which the steam 2 enters the housing 22, a nozzle end 24 from which the steam 2 exits the housing 20, and a nozzle valve or stem 26 disposed at the nozzle end 24. An actuator means 30 is connected to the first housing 20 for controlling the exit rate or exit pressure of the first fluid 2 at the nozzle end 24. The actuator means 30 may be of the type manufactured by Fisher Controls U.S.A.

The fluid mixing apparatus 10 further comprises a second, mixing housing 40 coupled to the first housing 20 at the nozzle end 24 of the first housing 20. The second housing 40 includes a second inlet 42 through which the first fluid 4 enters the second housing 40, and a third inlet 44 through which the second fluid 6 enters the second housing 40. The inlets 42 and 44 are disposed downstream of the first inlet 22. As shown in FIG. 1, the second inlet 42 and third inlet 44 are disposed in a common plane and spaced apart radially from each other, most preferably directly opposite (i.e., 180º apart) about the central axis Y of the mixing apparatus 10. The second housing 40 defines a generally cylindrical mixing chamber 52 that in turn defines a flow passage extending along the axial length of the mixing chamber 52 from an entry end 54 of the mixing chamber 52 to an exit end 56 of the chamber 52. The nozzle valve 26 is movable by the actuator 30 between seated and unseated positions at the entry end 54 to control the flow rate of steam 2 into the mixing chamber 52.

The nozzle end 24 of the first housing 20 directs the steam 2 into the entry end 54 of the mixing chamber 52. The second inlet 42 and the third inlet 44 radially direct the first fluid 4 and second fluid 6, respectively, into the mixing chamber 52. The steam 2, first fluid 4 and second fluid 6 are mixed in the mixing chamber 52 to form a molten mass or mixture 8 which exits the mixing chamber 52. The molten mass 8 then may be shaped into a shaped article or formed into a film, such as by casting the mixture 8 onto a cooling drum or by passing the mixture 8 through an extruder.

The fluid mixing apparatus 10 is adapted to produce a mixture for forming a film, more particularly an edible film for making edible strips or delivery systems. Incompatible film components generally are placed in different fluid inlet streams so that such incompatible components come together in the first instance at the interface of the steam injection within the mixing chamber 52 of the fluid mixing apparatus. While FIG. 1 shows inlets for steam, and first and second fluids, one or more additional inlets for one or more additional fluids may be provided. Preferably, the housings 20, 40 and other components of the fluid mixing apparatus 10 are constructed of high-grade stainless steel.

The gel film can also be used to modify the dissolution profile of the delivery system. For example, gel films of the invention can create delivery systems that have rapid dissolution in the mouth or that do not dissolve readily in the mouth allowing for a sustained delivery of the active substance over time. The delivery systems can have immediate release, enteric and delayed release capabilities once ingested. Definitions of "immediate release", "delayed release" and "enteric" can be found in the U.S. Pharmacopeia and are incorporated herein by reference.

The present invention is now described in more detail by reference to the following examples, but it should be understood that the invention is not construed as being limited thereto. Unless otherwise indicated herein, all parts, percents, ratios and the like are by weight.

### EXAMPLES

Unless otherwise indicated, the following procedures were used to prepare and evaluate the materials and films in Examples 1-4. The Stephan UMC5 processor is a laboratory scale mixing device which provided suitable high shear mixing, heating, and de-aerating of the formulations which were cast as films in the laboratory. A suitable batch size used with the Stephan UMC5 processor was 1500 grams.

An aqueous starch dispersion was prepared by dissolving any salts/buffers and pH modifiers in deionized water. The starch and/or maltodextrin (M100) were added and mixed until dissolved/dispersed. Pure Cote® B760 and B790 starches are available from the Grain Processing Corporation of Muscatine, Iowa.

A hydrocolloid mixture was prepared in the Stephan UMC5 processor by premixing the plasticizers until uniform, and adding the preblended dry hydrocolloids portionwise while mixing for about 30 second at 200 rpm after each addition. Sorbitol Special and glycerin were used as plasticizers. Sorbitol Special is an aqueous solution of sorbitol and sorbitol anhydrides at 76% solids supplied by SPI Polyols, Inc (New Castle, Delaware).

The starch dispersion was added to the non-aqueous hydrocolloid mixture and mixed at 300 rpm for 5 minutes. The mechanical agitation was increased to 2100 rpm and the mixture was heated to 85 °C to 95 °C with mixing. When the target temperature was achieved, the mixture was stirred for 30 minutes, then the sample was held under vacuum (50-60 bars) with continued agitation for an additional 45 minutes.

When the hold time under vacuum at temperature has been completed, the sample was poured into a preheated wide mouth quart Mason jar. Temperature and pH were recorded. Viscosity was measured on the hot sample using a Brookfield LVF viscometer.

A small portion of the sample was set aside and refrigerated usually overnight prior to measurement of gel/melt properties and solids using an Atago E series hand held refractometer (Gardco, Pompano Beach, FL). The melt temperature was determined by placing a small chunk of the refrigerated gel on a wire string stand held within a test tube so that the chunk does not contact the wall of the test tube. The test tube was covered with aluminum foil with a small hole to allow measurement of the gel temperature using a digital Tempermeter probe. The test tube was immersed in the heating bath so that the chunk is below the surface of a hot water bath at approximately 100 ^{o} C. A silicone oil bath was used for samples that had melt temperatures above 90 ^{o} C. The melt temperature was recorded when the gelled sample became wet in appearance, softened and could be stirred (a temperature range was noted). Once the sample had melted, the test tube was transferred to a second beaker containing cold tap water (15 ^{o} C). The temperature probe was used to record the temperature as the sample was cooled and to probe the sample surface to determine whether the sample had begun to gel. The gel temperature was the temperature upon cooling where the sample no longer flowed to fill in an indentation made by the probe.

The hot sample was then cast, using a draw down bar with a gap set to give a clearance of 3mm, onto 177 mm by 177 mm by 5 mm metal plates which were pre-sprayed with PAM (lecithin) to facilitate easy removal of film material. The gel coated plates were covered to avoid loss of moisture from the cast film. Cast films were typically refrigerated (less than 8 ^{o} C) for at least one-half hour prior to removal of the film for testing. Refrigeration is not required for film formation. Dried film strips were prepared by drying the coated plates in a 40 °C forced air/fan oven. Films dried 2 hours at 40 ^{o} C gave an intermediate solids of about 60%, while films dried overnight at 40 ^{o} C gave solids of 80% or higher. Test properties were measured at room temperature (approximately 20 °C) unless otherwise specified. The percent of solids of the dried film was determined between the cast film at its formulated solids level and the dried film by difference in weight. Break force (BF) was measured on the cast and dried film strips using a Texture Analyzer TA-108S Mini Film Test Rig.

Unless otherwise indicated, Maltrin M100 was obtained from Grain Processing Corporation, Pure-Cote B760 was obtained from Grain Processing Corporation, Sorbitol Special was obtained from SPI Polyols and Glycerin was obtained from VWR (EP/USP grade).

Example 1

As set forth below, Cgn A was obtained as an alkali processed, clarified extract of Gigartina skottsbergii , essentially haploid (gametophyte) plants and recovered by precipitation with alcohol. Minor levels (under 5% total) of lambda- and theta-carrageenans from diploid (tetrasporophyte) plants were also present.

Cgn B was obtained by dissolving Cgn A in water, and recovered by alcohol precipitation and drying. Samples of different molecular weights were obtained by reaction of the dissolved carrageenan with an oxidizing agent to yield Cgn C-F. Sodium hydroxide was added to samples Cgn C-E after the oxidation step and prior to alcohol precipitation to control the pH of the resulting product. The properties of the kappa-2 carrageenans are shown in Table 1. Viscosity of an aqueous solution at 1.5 wt % solids was measured at 75 ^{o} C using a Brookfield LVF viscometer at appropriate speeds and spindles. The properties of 2% water gels prepared using 2 wt% of samples Cgn A-F (#1) without added cations, (#2) with 0.2 wt% added KCl and (#3) with 0.2% added KCl and 0.2% CaCl ₂ , respectively, were characterized using a TXTM Texture Analyzer. Gels were tested at 25 ^{o} C and the break force (in grams) and the penetration (in centimeters) was recorded.

Cgns A-F below are examples of the kappa-2 carrageenans that can be used in the present invention.

Table 1: Properties of Kappa-2 Carrageenans A-F

| Test | Cgn A | Cgn B | Cgn C | Cgn D | Cgn E | Cgn F |
|---|---|---|---|---|---|---|
| Oxidization Treatment | No | No | Yes | Yes | Yes | Yes |
| Mg, % | 0.11 | 0.34 | 0.19 | 0.19 | 0.19 | 0.19 |
| Ca, % | 0.34 | 0.29 | 0.34 | 0.39 | 0.52 | 0.40 |
| K, % | 12.9 | 8.46 | 8.59 | 8.87 | 8.74 | 8.95 |
| Na, % | 0.22 | 0.42 | 0.51 | 0.57 | 0.65 | 0.38 |
| Visc, mPs* | 175 | 144 | 48 | 24 | 14 | 11 |
| PH | 9.4 | 9.42 | 8.93 | 9.03 | 9.16 | 6.7 |
| 2% water gel | | | | | | |
| BF (g) | 211 | 38 | 21 | 18 | 11 | 12 |
| Penetration (cm) | 7.4 | 13.9 | 11.5 | 9.0 | 7.8 | 16.1 |
| 2% water gel (KCl) | | | | | | |
| BF (g) | 308 | 162 | 126 | 107 | 70 | 51 |
| Penetration (cm) | 7.4 | 9.9 | 7.8 | 7.6 | 7.3 | 6.1 |
| 2% water gel (KCl + CaCl₂₎ | | | | | | |
| BF (g) | 487 | 349 | 514 | 445 | 356 | 158 |
| Penetration (cm) | 3.6 | 3.1 | 6.4 | 5.3 | 5.0 | 1.7 |

*VISCOSITY MEASURED IN 1.5% SOLIDS IN DEIONIZED WATER AT 75°C

In Table II below, Cgns D and E were formulated as shown and cast as films. The formulations and film properties are reported in Table II. All formulations are considered to be within the scope of the present invention, though some may be more preferable for a particular use than another.

Table II. Kappa-2 Carrageenan Formulations and Film Properties

| | Ex 1-1 | Ex 1-2 | Ex 1-3 | Ex 1-4 |
|---|---|---|---|---|
| Ingredients(g) | | | | |
| Water | 834.7 | 834.7 | 666 | 497.4 |
| Cgn D | 0 | 75 | 75 | 75 |
| Cgn E | 75 | 0 | 0 | 0 |
| M-100 | 227.3 | 227.3 | 292.3 | 357.2 |
| SORBITOL SP | 272.2 | 272.2 | 349.9 | 427.7 |
| GLYCERIN | 90.8 | 90.8 | 116.8 | 142.7 |
| Temp, ° C.* | 81.1 | 82 | 85 | 92 |
| VISCOSITY, MPAS* | 4000 | 13,700 | 22,350 | >50,000 |
| Solids (est) | 40.1% | 40.1% | 50.1% | 60.0 |
| Gel, ° C. | 55-57 | 54-55 | 62 | 77-78 |
| Melt, ° C. | 73-75 | 77-80 | 85 | 90-92 |
| As cast film | | | | |
| BF (g) | 312 | 318 | 404 | 476 |
| Dried film | | | | |
| (estimated 80% solids) | | | | |
| (16 hours @ 40° C.) | | | | |

*Temperature and viscosity of the molten mass prior to casting

The above Table shows that in Example 1-2 and Example 1-1, the viscosity of the molten mass at processing temperature (13,700 mPas and 4000 mPas, respectively) was controlled by decreasing the molecular weight of Cgn D to Cgn E (expressed as viscosity of 24 mPas and 14 mPas, respectively) with an insignificant impact on film properties.

The melt temperature of the cast material increased (Examples 1-2, 1-3 and -4) as the solids content was increased for a given formulation. In Examples 1-2, 1-3 and 1-4, the gel temperature increased with increasing solids until the gel temperature approached the temperature of the molten mass. Gelation, prior to casting, as indicated by the decreased gel strength of the cast film and the high molten state viscosity (>50,000 mPa) in Example 1-4, is due to the gel temperature approaching the temperature of the molten mass. This indicates the desirability of maintaining the temperature of the molten mass above the gelling temperature during processing if stronger films are desired. Agitation below the gel temperature results in a broken gel structure and decreased strength. Process equipment suitable for use adequately hydrate, homogeneously mix, and easily transport the molten mass for further processing "as is" or to additional operations, such as shaping or film casting.

Example 2

Kappa-2 carrageenan was obtained as an alkali processed, clarified extract of a mixture of Gigartina skottsbergii and Sarcothalia crispata , primarily haploid (gametophyte) plants. About 10-20% (total) of lambda- and theta-carrageenans from diploid (tetrasporophyte) plants was also present. The extract was recovered and subsequently ion exchanged to provide a kappa-2 carrageenan with low divalency. Properties of the low divalent cation kappa-2 carrageenans (Cgn G-J) are shown in Table III. Cgn G-J are considered to be within the scope of the invention.

Table III: Properties of Low Divalent Cation Kappa-2 Carrageenans

| | Cgn C | Cgn H | Cgn I | Cgn J |
|---|---|---|---|---|
| Cation Exchange | Yes | YES | YES | Yes |
| Mg, % | 0.07 | 0.02 | 0.03 | 0.05 |
| Ca, % | 0.06 | 0.01 | 0.16 | 0.15 |
| K, % | 2.19 | 1.00 | 0.00 | 0.67 |
| Na, % | 5.12 | 7.70 | 6.90 | 7.40 |
| Visc, mPs | 6 | 18 | 45 | 98 |
| PH | 8.12 | 8.7 | 9.6 | 10.1 |
| 2% water gel | | | | |
| BF (g) | 0 | 0 | 0 | 0 |
| 2% water gel (KCl) | | | | |
| BF (g) | 0 | 13 | 29 | 38 |
| 2% water gel (KCl + CaCl₂₎ | | | | |
| BF (g) | 30 | 93 | 112 | 181 |

Film compositions using the low divalent cation content kappa-2 carrageenans of samples Cgn G-J and the corresponding film properties are presented in Table IV. All formulations below are considered to be within the scope of the present invention, though some may be more preferable for a particular use than another.

Table IV: Films Using Low Divalent Cation Kappa-2 Carrageenan

| | Ex 2-1 | Ex 2-2 | Ex 2-3 | Ex 2-4 |
|---|---|---|---|---|
| Ingredients (g) | | | | |
| Water | 834.7 | 834.7 | 834.7 | 834.7 |
| Cgn J | 75 | 0 | 75 | 75 |
| Cgn I | 0 | 75 | 0 | 0 |
| KCl | 0 | 0 | 9.0 | 9.0 |
| STARCH B790 | 0 | 0 | 0 | 227.3 |
| M-100 | 227.3 | 227.3 | 227.3 | 0 |
| SORBITOL SP | 274.9 | 274.9 | 274.9 | 274.9 |
| GLYCERIN | 91.7 | 91.7 | 91.7 | 91.7 |
| Temp, ° C.* | 89 | 87 | 87 | 87 |
| VISCOSITY, MPAS* | 5800 | 5800 | 6250 | 10,300 |
| Solids (estimated) | 40% | 40% | 41% | 40% |
| Melt, ° C. | 45-48 | 43 | 66-71 | 70 |
| Gel, ° C. | 35 | 31 | 52 | 48 |
| As Cast Film | | | | |
| BF (g) | <40 | <40 | 281 | 237 |
| Dried film | | | | |
| Solids, | | | | |
| (estimated.˜80%) | | | | |
| Avg film thickness (mm) | – | – | 0.97 | 0.88 |
| BF (g) | 3468 | 3697 | 3236 | 7603 |

* TEMPERATURE AND VISCOSITY OF THE MOLTEN MASS PRIOR TO CASTING

The ion exchanged kappa-2 carrageenans (I and J) combined with polyols and maltodextrin (as a bulking agent) provided a relatively weaker cast gel film with negligible break force at 40% solids. This is believed to be due to insufficient amounts of potassium cations that are desired to more fully promote carrageenan double helix formation (i.e., gelling) at temperatures that allow the carrageenan to be the primary structuring agent. Examples 2-1 and 2-2 are gel films having relatively lower melt and gel temperatures. Even though gelation potential is not maximized (due to lower potassium levels), Examples 2-1 and 2-2 show a break force of 3468 and 3697, respectively. Example 2-3 demonstrates the effect of potassium ion addition to the structure formed by the kappa-2 carrageenan in Cgn J. Cast strength, although soft, provided sufficient strength for film removal from the casting plate. Structure development by Cgn J, with the addition of potassium ions, is confirmed by the increase in melt and gel temperatures in Example 2-3 as compared to Example 2-1. Break force of the dried film remained comparable to Examples 2-1 and 2-2.

Example 2-4 demonstrates the effect of replacing maltodextrin in Example 2-3 by a modified starch (B790). While viscosity was increased, the gel and melt temperatures remained relatively similar to Example 2-3 which contained the maltodextrin. The cast film strength of Example 2-4 was also relatively equal to Example 2-3. The dried film strength of Example 2-4 was more than doubled as compared to Example 2-3. This clearly indicates the structural synergy between the starch and kappa-2 carrageenan, when both are present with potassium cations (i.e., gelling ions for kappa-2 carrageenan). Potassium ions may be provided by direct addition of inorganic salts, organic salts, or combinations thereof or contained within additional ingredients. Use of kappa-2 carrageenan containing residual processing salts can promote the desired gel formation conditions that maximize gel structure and starch synergy. A homogeneous kappa-2 carrageenan/starch gel structure was formed by casting the molten mass at sufficiently high temperatures to prevent pre-gelation.

Additional formulations of the present invention are provided immediately below.

Table V: Kappa-2 Carrageenan Films

| Igredients (g) | Ex 2-5 | Ex 2-6 | Ex 2-7 |
|---|---|---|---|
| Water | 834.7 | 834.7 | 825 |
| Cgn H | 61.4 | 0 | 0 |
| Cgn G | 0 | 0 | 90 |
| Cgn I | 0 | 75.0 | 0 |
| Calcium sulfate dihydrate | 1.7 | 0 | 0 |
| Potassium chloride | 0 | 15.0 | 0 |
| STARCH B760 | 0 | 0 | 225 |
| M-100 | 227.3 | 227.3 | 0 |
| SORBITOL SP | 275.4 | 272.2 | 272.2 |
| GLYCERIN | 91.9 | 90.8 | 90.8 |

| | | | |
|---|---|---|---|
| Temp, ° C* | 82 | 88 | 75 |
| VISCOSITY, MPAS* | 6,500 | 16,150 | 18,250 |
| Solids, estimated | 39 | 42 | 40 |
| Melt, ° C. | 74-77 | 85 | 62-65 |
| Gel, ° C. | 56 | 60-65 | 42 |
| PH | 5.8 | 6.9 | 6.9 |
| AS CAST FILM | | | |
| BF (G) At ˜40% | 338 | 302 | 112 |
| Dried 2 hrs @ 40 C. | | | |
| BF (g) at ˜60% | 766 | NT | 536 |
| Dried overnight 16 hours @ 40° C. | | | |
| Avg film thickness | | 0.62 | |
| BF (g) at ˜80% | 3227 | 4470 | 6073 |

NT = not tested

* Temperature and viscosity of the molten mass prior to casting

Example 2-5 was prepared to have equivalent cation content with example 1-1. Both samples show the same gel melt properties. The higher molecular weight of Cgn E (14 cps) in Example 1-1 provided more structural support to the gel film as compared to Cgn H (6 cps) in Example 2-5, as is shown by the higher break force of the dried film. The higher dried film strength of the Example 2-7 shows that use of modified starch in combination with low molecular weight kappa-2 carrageenan provides overall film structure and indicates complexation of kappa-2 carrageenan with the starch.

Example 3

Table VI presents film formulations and properties of films containing kappa-2 carrageenans blended with alginates. KAHG is a potassium alginate having a high level of guluronic (G) units. Protanal® ester BV4830 is a propylene glycol alginate available from FMC BioPolymer (Philadelphia, PA).

Table VI: Kappa-2 Carrageenan and Alginate Blends

| | Ex 3-1 | Ex 3-2 |
|---|---|---|
| Ingredient | | |
| WATER | 55.6% | 55.6% |
| Cgn G | | |
| CGN C | 2.7% | 3.6% |
| KAHG | 2.1% | |
| BV4830 | 1.2% | 2.4% |
| STARCH B760 | | |
| M-100 | 15.0% | 15.0% |
| SORBITOL SP | 18.0% | 18.0% |
| GLYCERIN | 6.0% | 6.0% |
| Temp, ° C.* | 87 | 84 |
| VISCOSITY, MPAS* | 4250 | 1050 |
| Solids | 40 | 37 |
| Melt, ° C. | 77-78 | 74-79 |
| Gel, ° C. | 54 | 52 |
| PH | 4.8 | 5.5 |
| Cast film | | |
| (estimated 40%) | | |
| BF (g) | 142 | 168 |
| Dried film | | |
| (estimated 80%) | | |
| Avg film thickness (mm) | 0.62 | 0.48 |
| BF (g) | 3409 | 4004 |

* TEMPERATURE AND VISCOSITY OF MOLTEN MASS PRIOR TO CASTING

In example 3-1, potassium ion was supplied by the potassium alginate. Example 3-2 shows that propylene glycol alginate adds to strength to the kappa-2 carrageenan and lowers the processing viscosity.

Example 4

Table VII presents film produced using blends of Kappa-2 carrageenan with Edicol ULV 50, a low viscosity guar from Indian Gum Industries.

Cgn K was a kappa-2 carrageenan obtained as an alkali processed, clarified extract of Gigartina skottsbergii , essentially haploid (gametophyte) plants. Minor levels (under 5% total) of lambda- and theta-carrageenans from diploid (tetrasporophyte) plants were also present. Cgn K has a low divalent cation content and low potassium cation content as shown in Table I.

Cgn L was a kappa-2 carrageenan obtained as an alkali processed, clarified extract of a mixture of Gigartina skottsbergii and Sarcothalia crispata , primarily haploid (gametophyte) plants. About 10-20% (total) of lambda- and theta-carrageenans from diploid (tetrasporophyte) plants were also present. The properties of Cgn K and L are as follows.

Table VII: Properties of Kappa-2 Carrageenan

| | Cgn K | Cgn L |
|---|---|---|
| Cation Exchange | Yes | No |
| Mg, % | 0.05 | 0.05 |
| Ca, % | 0.15 | 0.45 |
| K, % | 0.67 | 13.40 |
| Na, % | 7.40 | 0.90 |
| Visc, mPs | 98 | NT |
| PH | 10.1 | NT |
| 2% water gel | | |
| BF (g) | 0 | ″ |
| 2% water gel (KCl) | | |
| BF (g) | 38 | ″ |
| 2% water gel (KCl + CaCl2) | | |
| BF (g) | 181 | ″ |

Table VIII shows the compositions and the film properties for formulations prepared using low viscosity guar gum in combination with kappa-2 carrageenans.

TABLE VIII: FORMULATIONS AND FILMS USING GUAR WITH KAPPA-2 CARRAGEENAN

| | Ex 4-1 | Ex 4-2 | Ex 4-3 | Ex 4-4 |
|---|---|---|---|---|
| Ingredients (g) | | | | |
| Water | 836.3 | 836.3 | 836.3 | 836.3 |
| Cgn L | 40.5 | 20.3 | 0.0 | 0.0 |
| Cgn K | 0.0 | 20.3 | 40.5 | 40.5 |
| GUAR ULV 50 | 49.5 | 49.5 | 49.5 | 49.5 |
| Starch B760 | 220.8 | 220.8 | 220.8 | 220.8 |
| POTASSIUM- | 0.0 | 0.0 | 4.5 | 4.5 |
| CHLORIDE | | | | |
| SORBITOL SP | 264.4 | 264.4 | 264.4 | 264.4 |
| GLYCERIN | 88.2 | 88.2 | 88.2 | 88.2 |
| Total weight (g) | 1500.0 | 1500.0 | 1500.0 | 1500.0 |
| Temp, ° C.* | 90 | 90 | 87 | 95 |
| VISCOSITY, MPA-S* | >50,000 | >50,000 | >50,000 | >50,000 |
| As cast | | | | |
| Gel, ° C. | 68-69 | 69 | 50 | 54-65 |
| Melt, ° C. | 85-87 | 86-88 | 67-68 | 76-83 |
| PH | 5.8 | 5.9 | 5.2 | 5.2 |
| Cast film | | | | |
| Solids, est | 45% | 42% | 40.2% | 45% |
| BF (g) | 239 | 349 | 130 | 330 |
| Dried film (2 hr 40° C.) | | | | |
| Solids, est | 60% | 60% | 63% | 66% |
| BF (g) | 953 | 2189 | 1194 | 1631 |
| Dried film | | | | |
| (16 hr, 40° C.) | | | | |
| Solids, est | 87% | 75% | 84% | 84% |
| BF (g) | 7476 | 6901 | 6276 | 8733 |

* Temperature and viscosity of the molten mass prior to casting.

Example 5

The following examples show films made using the fluid mixing apparatus of Figure 3. In these examples, Part A and Part B were pumped from separate holding tanks at ambient temperature, as two separate streams 4, 6, into two different inlet ports 42, 44 which fed the steam injection fluid mixing apparatus device 10. The two individual streams 4, 6 were combined at the interface of the steam in the mixing zone 52 of the fluid mixing apparatus 10. The separate solutions of Part A and Part B were readily pumped into the fluid mixing apparatus 10 and mixed with steam 2. The steam 2 was introduced to the mixing zone at a pressure of 120 psi. The resulting molten mass or slurry mixture 8 flowed out of the exit port 56 of the fluid mixing apparatus 10. The mixture 8 was poured onto a smooth surface and drawn down to form a homogeneous film 9.

To measure the viscosity of the mixture 8, approximately 500 ml sample of the mixture 8 was collected from the outlet 56 and poured into a jar. The temperature, pH and viscosity were measured for this sample at 95ºC. A Brookfield LVF viscometer was used to measure the viscosity. An appropriate speed and spindle combination were used such that a reading could be taken. The dial reading was converted to dynamic viscosity (cP).

To measure the film strength and solids level, the molten mass 8 was collected from the outlet 56 then cast using a draw down bar, with a gap set at 3 mm, onto a stainless steel metal plate. The initial films 9 or "fresh films" were collected. Portions of the fresh films 9 were dried by placing them in a 40ºC forced air oven. Break force was measured on the fresh and dried film strips using a Texture Analyzer TA-108S Mini Film Test Rig. The percent solids were determined by measuring the difference between the initial weight of the fresh film and the final weight of the dried films.

To measure the gel temperature, a portion of the molten mass 8 was collected from the outlet 56 of the mixing apparatus 10 and transferred to a test tube. Half of the test tube remained empty. A glass thermometer was inserted into the molten mass 8. The material 8 was allowed to cool under room temperature conditions. After each degree of cooling, the thermometer was removed from the material 8. When a small, temporary indentation was observed in the surface of the mass 8, this temperature was recorded. The thermometer was re-inserted into the mass 8, which was allowed to cool further. The thermometer was removed and re-inserted at every degree of cooling until such a time as a permanent indentation formed in the mass 8, such that the indentation did not refill. The temperature at which the permanent indentation formed was recorded. The gel temperature reported was the range between the two recorded temperatures.

Table IX - Mixtures Containing Kappa-2 Carrageenan

| Example No. | 4A | 4B | 4C |
|---|---|---|---|
| Part A (%) | | | |
| Kappa-2 Carrageenan A | 7.0 | 8.4 | 8.9 |
| Glycerin | 26.5 | 31.8 | 33.5 |
| Part B (%) | | | |
| Starch | 16.4 | 19.7 | 20.7 |
| Water | 50.0 | 40.0 | 36.9 |
| Mixing chamber temp. (° C.) | 107 | 107 | 108 |
| Outlet temp (° C.) | 101 | 102 | 102 |
| Viscosity cP (@ 95° C.) | 7300 | 5200 | 48000 |
| pH | 7.3 | not tested | 8 |
| % solids | 53 | 54 | 65 |
| Gel temp. (° C.) | 46-50 | 43-47 | 53-60 |
| wet film strength (grams) | 267 | 214 | 983 |
| Dry film strength (grams) | 2958 | 6798 | 4594 |

Table X - Mixtures Containing Kappa-2 Carrageenan and PGA

| Example No. | 5-4 | 5-5 | 5-6 | 5-7 |
|---|---|---|---|---|
| Part A (%) | | | | |
| Carrageenan A | 2.7 | 3.2 | 3.2 | 4.0 |
| PGA | 3.3 | 3.9 | 3.9 | 4.9 |
| Glycerin | 22.4 | 26.5 | 26.5 | 33.5 |
| Part B (%) | | | | |
| KOH | 0.0 | 0.0 | 0.1 | 0.0 |
| K_{2CO} | 0.0 | 0.0 | 0.0 | 0.3 |
| Starch | 13.9 | 16.4 | 16.4 | 20.7 |
| Water | 57.8 | 50.0 | 49.9 | 36.6 |
| Mixing chamber temp. (° C.) | 108 | 107 | 108 | 107 |
| Outlet temp (° C.) | 102 | 102 | 102 | 101 |
| Viscosity Cp (@ 95° C.) | 5500 | 4650 | 2200 | 12400 |
| pH | 4.1 | 4.2 | 8.7 | 6.3 |
| % solids | 48 | 50 | not tested | 58 |
| Gel temp. (° C.) | 35-40 | Not tested | not tested | 58-66 |
| Wet film strength (grams) | 60 | 117 | not tested | 337 |
| dry film strength (grams) | 2408 | 3069 | 4335 | 4561 |

Table XI - Mixtures Containing Kappa-2 Carrageenan and LV Guar

| Example No. | 5-8 | 5-9 |
|---|---|---|
| Part A (%) | | |
| Carrageenan B | 4.0 | 4.2 |
| ULV guar | 4.9 | 5.1 |
| Glycerin | 33.5 | 27.0 |
| Sorbitol | 0.0 | 8.1 |
| Part B (%) | | |
| Starch | 20.7 | 21.8 |
| Water | | |
| Mixing chamber temp. (° C.) | 108 | 108 |
| Outlet temp (° C.) | 102 | 102 |
| Viscosity cP (@ 95° C.) | 7800 | 69000 |
| PH | 5.6 | 5.5 |
| % solids | 57 | 55 |
| Gel temp. (° C.) | >100 | >100 |
| Wet film strength (grams) | 3402 | 921 |
| Dry film strength (grams) | 6587 | 9234 |

The following Tables XII and XIII provide further descriptions of the components specified in this example.

Table XII - Component Descriptions

| | | | |
|---|---|---|---|
| Name | Trade Name | Supplier | Description |
| Propylene glycol | Protanal BV | FMC Corporation | |
| alginate (PGA) | 4830 | | |
| low viscosity | Edicol ULV 50 | Indian Gum | |
| guar (LV guar) | | Industries, Ltd | |
| Glycerin | | Callahan | 99.70% |
| | | Chemical | |
| Sorbitol | Sorbo | SPI Polyols | 70% sorbitol |
| | | | solution, |
| | | | USP/FCC |
| starch | Pure-Cote B790 | Grain Processing | |
| | | Corporation | |

Table XIII - Carrageenan Descriptions

| | | |
|---|---|---|
| Reference | Description | Supplier |
| Kappa-2 | An alkali processed, clarified, low divalency | FMC |
| Carrageenan | extract of a mixture of Gigartina skottsbergii | Corporation |
| A | and Sarcothalia crispata, primarily haploid | |
| | (gametophyte) plants, such extract being | |
| | commonly known as “kappa-2 carrageenan”. | |
| | Includes about 10-20% (total) of lambda | |
| | and theta-carrageenans from diploid | |
| | (tetrasporophyte) plants. Defined as the | |
| | natural random block copolymer of kappa- | |
| | and iota-carrageenan in the ratio of about | |
| | 1.0 to 3.0:1 respectively, and has | |
| | significantly different functionalities | |
| | compared to mixing individual kappa and | |
| | iota-carrageenan natural polymers at the | |
| | same ratio. | |
| Kappa-2 | An alkali processed, clarified, low divalency | FMC |
| Carrageenan | extract of Gigartina skottsbergii, essentially | Corporation |
| B | haploid (gametophyte) plants, such extract | |
| | being commonly known as “kappa-2 | |
| | carrageenan”. Also includes minor levels | |
| | (under 5% total) of lambda- and theta- | |
| | carrageenans from diploid (tetrasporophyte) | |
| | plants. Defined as the natural random block | |
| | copolymer of kappa- and iota-carrageenans | |
| | in the ratio of about 1.0 to 3.0:1 respectively, | |
| | and has significantly different functionalities | |
| | compared to mixing individual kappa- and | |
| | iota-carrageenan natural polymers at the | |
| | same ratio. | |

Example 6

Kappa-2-carrageenan (ion exchanged/sodium) delivery films were prepared by the following method: Maltrin M 100 maltodextrin (Grain Processing Corp., Muscatine, Iowa) was dry blended with the kappa-2-carrageenan. Deionized water and glycerin were weighed into a 1.2 liter stainless steel beaker. The dry premix was added to the water with good agitation, then heated to 90 ^{o} C and held within the temperature range 90 ^{o} C to 95 ^{o} C for 15 minutes to fully hydrate the gums. After replacing any water loss due to evaporation, the delivery ingredient was added and mixed two minutes for dispersion. The delivery ingredients tested were: (1) natural and artificial strawberry flavor (Dragoco, 0.1%), (2) titanium dioxide, and (3) caffeine. The hot solution was quickly poured into containers. Solution cast in petri dishes was cooled to room temperature to form films, then dried overnight in a forced draft oven at 45 ^{o} C to a constant weight. A sample was cooled then refrigerated (less than 8 ^{o} C) overnight prior to measurement of gel/melt properties and solids using an Atago E series hand held refractometer (Gardco, Pompano Beach, FL). The melt temperature was determined by placing a small chunk of the refrigerated gel on a wire string stand held within a test tube so that the chunk did not contact the wall of the test tube. The test tube was covered with aluminum foil with a small hole to allow measurement of the gel temperature using a digital Tempermeter probe. The test tube was immersed in the heating bath so that the chunk is below the surface of a hot water bath at approximately 100 ^{o} C. A silicone oil bath was used for samples that had melt temperatures above 90 ^{o} C. The melt temperature was recorded when the gelled sample became wet in appearance, softened and could be stirred (a temperature range was noted). Once the sample had melted, the test tube was transferred to a second beaker containing cold tap water (15 ^{o} C). The temperature probe was used to record the temperature as the sample was cooled and to probe the sample surface to determine whether the sample had begun to gel. The gel temperature was the temperature upon cooling where the sample no longer flowed to fill in an indentation made by the probe. Break force (BF) and penetration were measured on the cast and dried film strips using a Texture Analyzer TA-108S Mini Film Test Rig. Rigidity was calculated as the BreakForce divided by the penetration.

Table XIV: Kappa-2 Carrageenan Delivery Systems

| | A | B | C |
|---|---|---|---|
| Kappa-2 | 3.0 | 3.0 | 3.0 |
| Maltodextrin | 7.0 | 7.0 | 10.0 |
| Water | 80.0 | 80.0 | 80.0 |
| Glycerin | 6.7 | 6.7 | 6.7 |
| Flavor | 3.3 | 0.0 | 0.0 |
| TiO2 | 0.0 | 3.3 | 0.0 |
| Caffeine | 0.0 | 0.0 | 0.3 |
| Cast Film | | | |
| (˜20% solids) | | | |
| BF, g | 145 | 150 | 100 |
| Penetration, cm | 1.3 | 1.0 | 1.3 |
| Rigidity | 112 | 153 | 74 |
| Dried film | | | |
| (˜80% Solids) | | | |
| BF (g) | 3417 | 9827 | 6374 |
| Penetration, cm | 1.8 | 1.8 | 1.5 |

As described and demonstrated above, the films made in accordance with this invention can be used to make delivery systems containing a variety of active substance.

While the invention has been described in detail and with reference to specific embodiments thereof, it will be apparent to one skilled in the art that various changes and modifications can be made therein without departing from the spirit and scope thereof.

1. A delivery system comprising a homogenous, thermoreversible gel film, wherein said gel film comprises: (i) a film forming amount of kappa-2 carrageenan and optionally at least one of a plasticizer, a second film former, a bulking agent, and a pH controlling agent; and (ii) an active substance.

2. The delivery system of claim 1, wherein said active substance is at least one of an oral care agent, a breath freshening agent, antimicrobial agent, cooling agent, a pharmaceutical agent, a nutraceutical agent, a salivary stimulant agent, cosmetic ingredient, agricultural active, a vitamin, a mineral, a coloring agent, a sweetener, a flavorant, a fragrance or a food.

3. The delivery system of claim 1, wherein said gel film further comprises at least one of potassium, sodium or ammonium cation in an amount less than 20% by dry weight of the kappa-2 carrageenan in the gel film.

4. The delivery system of claim 1, wherein said kappa-2 carrageenan is present in an amount of at least 0.5% by dry weight of the gel film.

5. The delivery system of claim 1, wherein said kappa-2 carrageenan is present in an amount of 0.5% to 25% by dry weight of the gel film.

6. The delivery system of claim 1, wherein said kappa-2 carrageenan is present in an amount of 1.5% to 25% by dry weight of the gel film.

7. The delivery system of claim 1, wherein said kappa-2 carrageenan is present in an amount of at least 10% of the total dry weight of film formers in the gel film.

8. The delivery system of claim 1, wherein said kappa-2 carrageenan is present in an amount of at least 20% of the total dry weight of film formers in the gel film.

9. The delivery system of claim 1, wherein said kappa-2 carrageenan is present in an amount of at least 50% of the total dry weight of film formers in the gel film.

10. The delivery system of claim 1, wherein said kappa-2 carrageenan is present in an amount of at least 80% of the total dry weight of film formers in the gel film.

11. The delivery system of claim 1, wherein said kappa-2 carrageenan is the only film former present in the gel film.

12. The delivery system of claim 1, wherein said second film former is selected from the group consisting of starch, starch derivative, starch hydrozylate, cellulose gums, kappa carrageenan, iota carrageenan, alginates, propylene glycol alginate, polymannan gums, dextran, pullulan, gellan, pectin, alkylcellulose ethers and modified alkyl cellulose ethers.

13. The delivery system of claim 1, wherein said plasticizer is at least one member selected from the group consisting of glycerin, sorbitol, maltitol, lactitol, corn starch, fructose, sucrose, and polyalkylene glycols; said second film former is at least one member selected from the group consisting of a starch, starch derivative, starch hydrozylate, cellulose gum, kappa carrageenan, iota carrageenan, alginates, propylene glycol alginate, polymannan gums, gellan, pullulan, dextran, pectin, an alkylcellulose ether and a modified alkyl cellulose ether; and said bulking agent is at least one member selected from the group consisting of microcrystalline cellulose, sugar, corn syrup, polydextrose, starch, starch derivatives, inulin, and starch hydrozylates.

14. The delivery system of claim 1, having a break force strength of at least 2,500 grams.

15. The delivery system of claim 1, having a break force strength of at least 4,000 grams.

16. The delivery system of claim 1, having a break force strength of at least 5,000 grams.

17. The delivery system film of claim 1, having a break force strength of at least 6,000 grams.

18. A process for preparing the homogeneous gel film delivery system in any of claims 1-17 comprising the steps of:
(i)heating, hydrating, mixing, solubilizing and, optionally, de-aerating said kappa-2 carrageenan and optionally at least one of a plasticizer, a second film former, a bulking agent, and a pH controlling agent in an apparatus providing sufficient shear, temperature and residence time to form a homogeneous molten composition, wherein said temperature is at or above the solubilizing temperature of said composition;
(ii)adding an effective amount of an active substance either prior to or after formation of the molten composition; and
(iii)cooling said molten composition containing said active substance at or below its gelling temperature to form said gel films containing said active substance.

19. The process of claim 18, wherein said active substance is at least one of an oral care agent, a breath freshening agent, an antimicrobial agent, a cooling agent, a pharmaceutical agent, a nutraceutical agent, a salivary stimulant agent, a vitamin, a mineral, a cosmetic ingredient, an agricultural active, a coloring agent, a sweetener, a flavorant, a fragrance, a food.

20. The delivery system of claim 1, having a break force strength of at least 250 grams.

21. The delivery system of claim 1, having a break force strength of at least 1,000 grams.

22. The process of claim 18, wherein said apparatus is a Ross mixer, Stephan processor, extruder, jet cooker or fluid mixing apparatus.

23. The delivery system of claim 1 having a solids content of at least 50%, wherein said kappa-2 carrageenan has an extract viscosity of less than 10 cps at 75 °C as measured in a 1.5% solids 0.10 molar sodium chloride solution.

24. The delivery system of claim 1, further comprising a flavorant and having a solids content of at least 50%.

25. The delivery system of claim 1, wherein said gel film does not contain a plasticizer.

26. The delivery system of claim 1 consisting of said kappa-2 carrageenan, a bulking agent, an active substance, and water.

27. The delivery system of claim 26, wherein said bulking agent is corn syrup.
